(19) United States
(12) Patent Application Publication
(10) Pub. No.: US 2008/0049893 A1
(43) Pub. Date: Feb. 28, 2008

(54) Rotary device for an optic tomography and optic tomography with a rotating device

(76) Inventors: Karlheinz BARTZKE, Jena (DE); Georg GUENTHER, Grossschwabhausen (DE); Ralf WOLLESCHENSKY, Jena (DE); Michael WEGWERTH, Jena (DE)

Correspondence Address: JACOBSON HOLMAN PLLC, 400 SEVENTH STREET N.W., SUITE 600, WASHINGTON, DC, 20004, US

(21) Appl. No.: 11/892,813

(22) Filed: Aug. 27, 2007

(30) Foreign Application Priority Data
Aug. 25, 2006 (DE) 10 2006 039 935.8

(51) Int. Cl. A47B 11/00; A61B 6/00; G01N 21/00

(52) U.S. Cl. 378/25; 108/20

(57) ABSTRACT

A rotary device (1) is provided for an optic tomograph, with the sample carrier (4) with the object (5) to be examined being rotational around a predetermined axis (6) of the object, with the rotary device (1) having a rotary module (8; 15), which can cause a rotation around a first rotary axis (10), a first positioning module (7) connected to the rotary module (8), by which the first rotary module (8; 15) can be positioned along the first rotary axis (10), and a second positioning module (9; 11; 12; 16; 17) connected to the rotary module (8; 15), at which the sample carrier (4) with the object (5) to be examined can be fastened, allowing the axis (6) of the object to be positioned in reference to the first rotary axis (10) by the second positioning module (9; 11, 12; 16; 17) such that a rotation of the rotary module (8; 15) rotates the object (5) around the axis (6) of the object.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A “SEQUENCE LISTING,” A TABLE, OR A COMPUTER PROGRAM

Not Applicable

### BACKGROUND OF THE INVENTION

(1) Field of the Invention

The present invention relates to a rotary device for an optic tomography and an optic tomography with a rotating device.

(2) Description of Related Art

In optic tomography, for example when fluorescent radiation of an object fixed in a gel rod is detected in various directions, a rotating device is necessary in order to find the object to be observed microscopically (e.g., an embryo in a gel rod) and to rotate it around an axis extending through the object with a precision of approx. 0.2 μm.

Therefore, the object of the present invention is to provide a rotating device for an optic tomography, by which the object can be rotated around a predetermined axis of the object with a predetermined precision. Further, an optic tomography shall be provided having such a rotating device.

### BRIEF SUMMARY OF THE INVENTION

The object of the invention is attained in a rotating device for optical tomography, with a sample carrier with an object to be examined being rotated around a predetermined axis of an object, with the rotating device being provided with a rotary module, which may cause a rotation around a first rotary axis, a first positioning module, which is connected to the rotary module and which may be positioned with the rotary module along the first rotary axis, and a second positioning module, which is connected to the rotary module and which can be mounted to the sample carrier with the object to be examined, allowing the axis of the object to be positioned in reference to the first rotary axis via the second positioning module such that the object is rotated around the axis of the object when the rotary module is rotated.

With this rotary device, using a relatively simple design, the necessary precision can be accomplished for rotation around the predetermined axis of the object. In particular, the rotary module can be provided with a first rotary table, which can be rotated around the first rotary axis, and the axis of the object can be positioned via the second positioning module such that it coincides with the first rotary axis. Further, the second positioning module may be provided with a x-y-table, which is connected to the rotary module in a torque-proof manner.

It is also possible for the second positioning module to be provided with a second and a third rotary table, with the second rotary table being mounted so that it rotates relative to the first rotary table, the third rotary table, rotational around a third rotary axis, being mounted to the second rotary table in a torque-proof fashion and neither the first and second rotary axis nor the second and third rotary axis coincide.

Further, the second positioning module may be provided with a second table, which is connected to the first rotary table via a linear displacement device, with the object carrier being rotational in reference to the first rotary table. The ability to rotate the object carrier, for example the second table, may be embodied rotational in reference to the first rotary table.

Further, the rotary module may be provided with a x-y-table, which is controlled such, that the second positioning module is rotated around the first rotary axis, and in which the second positioning module is provided with a rotary table, which is connected to the x-y-table of the rotary module in a torque-proof manner.

The object is further attained by an optic tomography having an above-described rotary device, a lighting module for illuminating the object, a detection module for detecting at least one optic cross-section of the object, and a control module for controlling the tomography. The detection module can for example detect fluorescent radiation of the object. Of course, each other radiation emitted by the object due to lighting may also be detected. In particular, the detection module may detect confocally the radiation of the object.

The optic tomography can in particular be embodied as a microscope or as a laser scanning microscope.

### BRIEF DESCRIPTION OF THE SEVERAL VIEWS OF THE DRAWINGS

In the following the invention is described in greater detail in an exemplary fashion using the attached drawings.

FIG. 1 is a schematic drawing in perspective of the optic tomography according to the subject invention;

FIG. 2 is a side view of the rotary device according to the first embodiment of FIG. 1;

FIG. 3 is a view of the rotary device of FIG. 2 from the bottom;

FIG. 4 is a view of a modification of the rotary device of FIG. 2 from the bottom;

FIG. 5 is a side view of another embodiment of the rotary device, and

FIG. 6 is a view of the modification of the rotary device of FIGS. 2 and 3 from the bottom.

### DETAILED DESCRIPTION OF THE INVENTION

In describing preferred embodiments of the present invention illustrated in the drawings, specific terminology is employed for the sake of clarity. However, the invention is not intended to be limited to the specific terminology so selected, and it is to be understood that each specific element includes all technical equivalents that operate in a similar manner to accomplish a similar purpose.

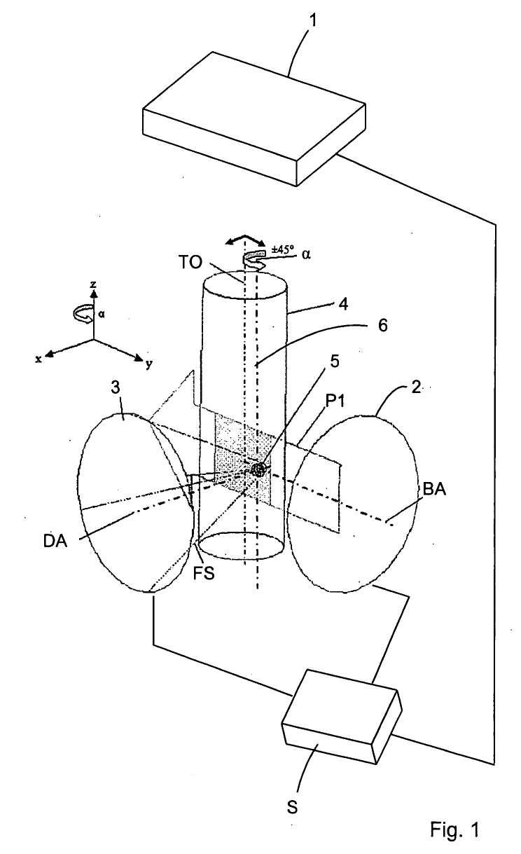

In FIG. 1, schematically an optic tomography is shown, which comprises a rotary device 1, a lighting module 2, a detection module 3, as well as a control module S. For the lighting module 2 and the detection module 3 only one lens for each is shown schematically. Of course, the lighting module 2 and the detection module 3 comprise additional elements known to those skilled in the art in order to allow the performance of illumination and detection necessary for optic tomography.

At the rotary device 1 a sample carrier 4 (here a gel rod) with an object 5 to be examined is fastened. For better visibility, the rotary device 1 is shown in an exploded representation in reference to the gel rod 4. Actually, the gel rod 4 is held by the rotary device 1. The object may, for example, represent an embryo 5 embedded in the gel rod 4.

The object 5 is lit via the lighting module 2 along a lighting axis BA, with the lighting module 2 emitting a linearly focused bundle of light beams (as indicated by the arrow P1) so that in the gel rod 4 and thus also in the embedded embryo 5 an area is lit positioned parallel to the y-z-level. The fluorescent radiation FS created this way in the embryo is detected via the detection module 3 so that an optic cross-section through the embryo 5 is detected (the detection axis is called DA). In particular, the detection can be performed confocally in order to yield a very good resolution in the x-direction.

The rotary device 1 is embodied such that it positions the gel rod 4 (in the z-direction) and rotates it (around an axis parallel in reference to the z-axis by at least the rotary angle α equaling ±45°) such that the gel rod 4 is rotational around an object axis 6 (parallel in reference to the z-axis) which passes through the object 5, independent from the position of the object 5 in the gel rod 4. Here, the axis 6 of the object is off-set parallel in reference to the central axis TO of the gel rod 4. This way, via the optic tomography, optic cross-sections can be detected in various rotational positions of the object 5, from which, then in a known fashion, the desired three-dimensional image of the object can be generated. The positioning, rotation of the object 5, lighting, detection and, if necessary, evaluation of the measuring data, and the image creation occur under the control of the control module S.

The detection module 3 can detect the object in an enlarged fashion so that the optic tomography may also be used as a microscope. The lighting module 2 and the detection module 3 may be embodied such that together with the rotary table, the control module S, and if necessary other modules known to those skilled in the art are assembled to form a laser-scanning microscope, by which in a known fashion optic cross-sections through an object 5 can be detected in various depths in the object. By the rotary device 1 these optic cross-sections can be performed in various depths in the object, depending on the various rotary positions, so that a high-resolution three-dimensional image of the object 5 can be produced.

In the embodiment shown in FIG. 1 the lighting axis BA and the detection axis DA form an angle amounting to 90°. Of course, other angles are also possible, in particular the lighting axis BA and the detection axis DA may coincide. Further, the optic tomography (and/or the microscope) may also be embodied such that the lighting module implements a top-light or a penetrating light.

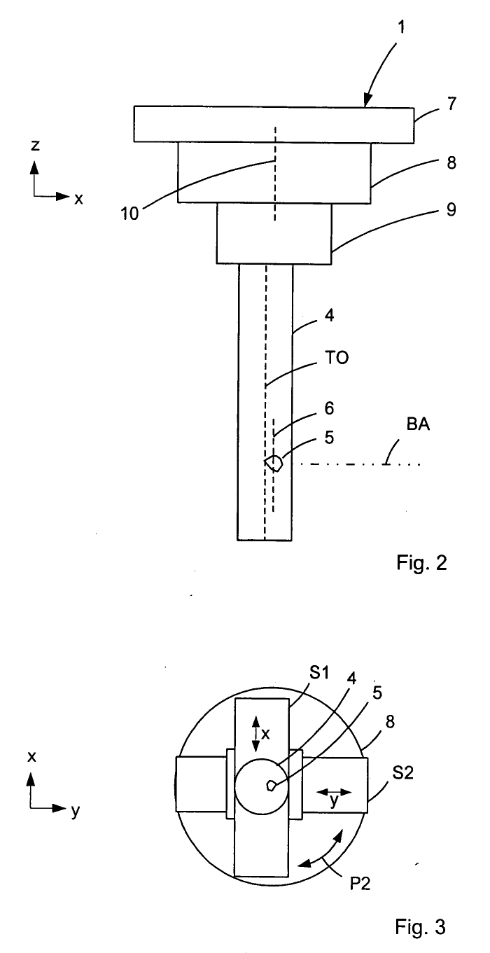

In the embodiment of the laser-scanning microscope, the bundle of illuminating radiation may not be embodied linearly, but for example also focused punctually into the object 5. Further, the lighting module 2 and the detection module 3 may be embodied such that the illumination and/or the detection occur confocally. In order to allow the realization of the desired rotation around the axis 6 of the object, with at least a rotation being desired of ±45°, the rotary device 1 may be embodied, for example, as shown in FIGS. 2 and 3.

The rotary device 1 comprises a first positioning module 7, with a first rotary table 8 being mounted to its bottom (FIG. 2). At the bottom of the first rotary table 8, an x-y-table 9 is connected to the first rotary table 8 in a torque-proof manner. At the x-y-table 9, the gel rod 4 is held in a torque-proof manner. The first positioning module 7 serves to allow an adjustment of the first rotary table 8 together with the x-y-table 9 and the gel rod 4 along the z-direction, so that the object 5 can be positioned in the desired z-position in reference to the lighting module 2 and the detection module 3. The x-y-table 9, provided with a first sled S1 for positioning in the x-direction and a second sled S2 for positioning in the y-direction, serves to position the axis 6 of the object such that it is aligned and/or coincides with the rotary axis 10 of the rotary table 8 (such as, for example, indicated in FIG. 3 in the view from the bottom). When the axis 6 of the object coincides with the rotary axis 10 a rotation of the first rotary table 8 (indicated by the arrow P2 in FIG. 3) leads to a rotation of the object 5 around the axis 6 of the object. Thus, using simple means the desired positioning and rotation of the object 5 can be achieved with the required precision.

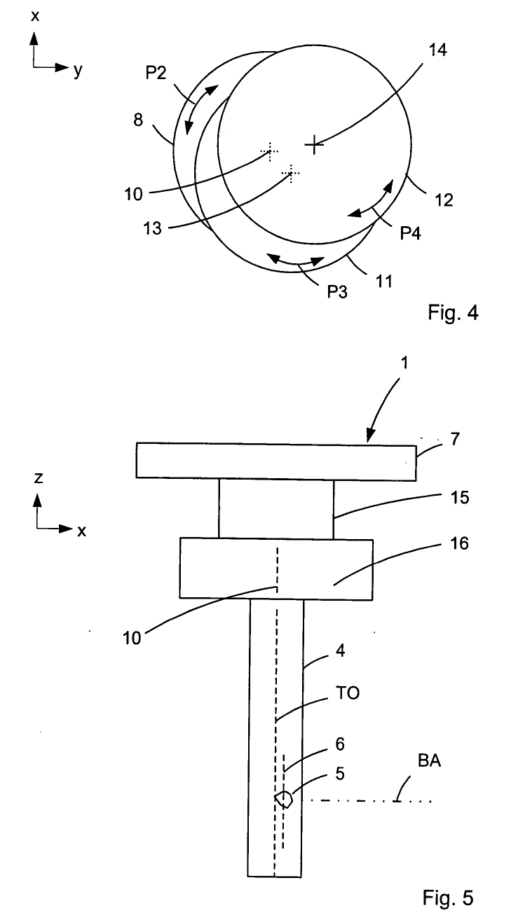

FIG. 4 shows a deviation of the rotary device of FIGS. 2 and 3. In the embodiment of FIG. 4, which shows a view of the rotary device 1 from the bottom, instead of the x-y-table 9 a second and third rotary table 11, 12 are provided. The second rotary table 11 is connected to the first rotary table 8, rotational around a second rotary axis 13, (indicated by the arrow P3). The third rotary table 12 is connected to the second rotary table 11 (rotational around a third rotary axis 14 (indicated by the arrow P4). The first and second rotary axes 10 and 13 are offset in reference to each other. The same applies for the second and third rotary axes 13 and 14 so that via the second and third rotary table 11, 12 again the axis 6 of the object of the gel rod 4 (in FIG. 4 the gel rod 4 is not shown to simplify the illustration) can be positioned such that it coincides with the first rotary axis 10 of the first rotary table 8. Thus, based on a rotation of the first rotary table 8, the object 5 is rotated around the axis 6 of the object.

FIG. 5 shows another embodiment of the rotary device. In this embodiment the rotary device comprises the first positioning module 7, an x-y-table 15 connected to the bottom of the first positioning module 7, as well as a rotary table 16, which is connected to the x-y-table 15 in a torque-proof manner. At the bottom of the rotary table 16 the gel rod 4 is held in a torque-proof manner. The first positioning module 7 serves in the same manner as in the embodiments of FIGS. 2 through 4 to position the object 5 in the z-direction.

The x-y-table 15 is addressed such that both in the x-direction as well as the y-direction a sinus-shaped motion is performed. This results in the rotary table 16 moving circularly together with the respectively centrally arranged gel rod 4. The gel rod 4 is simultaneously rotated around its central axis TO by the rotary table 16, with an entire rotation of the gel rod being performed per period of the sinus motion in the x and the y direction. Therefore, an arbitrary rotary axis can be set within the gel rod 4, which extends parallel to its central axis TO, as the rotary axis of the combined motion of the x-y-table 15 and the rotary table 16 so that again the axis 5 of the object can be selected freely in the gel rod 4.

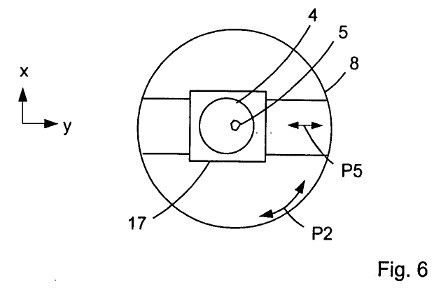

FIG. 6 shows another embodiment of the rotary device. This embodiment is based on the embodiment of FIGS. 2 and 3 and differs therefrom such that instead of the x-y-table 9 a linear table 17 is provided, which is connected to the first rotary table 8 such that it can execute only a linear motion in one direction (indicated by the double-arrow P5) in reference to the rotary table 8. The gel rod 4 is mounted rotational on this linear table 17 such that by the rotation of the gel rod 4 in reference to the linear table 17 and the linear motion of the linear table the axis 6 of the object can be positioned such that it coincides with the first rotary axis 10 of the first rotary table 8. Of course, the linear table 17 may also be embodied such that it can be rotated in reference to the first rotary table 8. In this case the gel rod 4 can be connected to the linear table 17 in a torque-proof manner.

Modifications and variations of the above-described embodiments of the present invention are possible, as appreciated by those skilled in the art in light of the above teachings. It is therefore to be understood that, within the scope of the appended claims and their equivalents, the invention may be practiced otherwise than as specifically described.

What is claimed is:

1. A rotary device for an optic tomograph that has a sample carrier with an object to be examined, the object being rotational around a predetermined axis of the object, the rotary device comprising:
a first rotary module for causing a rotation around a first rotary axis, a first positioning module, operatively connected to the rotary module, and by which the first rotary module is positioned along the first rotary axis, and a second positioning module, operatively connected to the rotary module and mounted to the sample carrier with the object to be examined, allowing the axis of the object to be positioned with reference to the first rotary axis by way of the second positioning module such that when the rotary module is rotated, the object is turned around the axis of the object.

2. The rotary device according to claim 1, in which the rotary module is provided with a first rotary table that can be rotated around the first rotary axis, and in which the axis of the object can be positioned by way of the second positioning module such that it coincides with the first rotary axis.

3. The rotary device according to claim 2, in which the second positioning module has an x-y-table, which is connected to the rotary module.

4. The rotary device according to claim 2, in which the second positioning module is provided with a second rotary table and a third rotary table with the second rotary table being mounted to the first rotary table and being rotational around a second rotary axis, the third rotary table at the second rotary table, being rotational around a third rotary axis, and the first and second rotary axes as well as the second and third rotary axes not coinciding with each other.

5. The rotary device according to claim 2, in which the second positioning module is provided with a second table, which is connected to the first rotary table by way of a linear displacement device, with the sample carrier being mounted at the second table and being rotational with respect to the first rotary table.

6. The rotary device according to claim 5, in which the second table is rotational with respect to the first rotary table.

7. The rotary device according to claim 1, in which the rotary module is provided with an x-y-table controlled such that the second positioning module is rotated around the first rotary axis and in which the second positioning module is provided with a rotary table that is connected to the x-y-table of the rotary module.

8. An optic tomograph for examining an object, the optic tomograph comprising:
a sample carrier carrying the object to be examined, the object being rotational around a predetermined axis of the object,
a first rotary module for causing a rotation around a first rotary axis,
a first positioning module, operatively connected to the rotary module, and by which the first rotary module is positioned along the first rotary axis,
a second positioning module, operatively connected to the rotary module and mounted to the sample carrier with the object to be examined, allowing the axis of the object to be positioned with reference to the first rotary axis by way of the second positioning module such that when the rotary module is rotated, the object is turned around the axis of the object,
a lighting module for illuminating the object,
a detection module for detecting at least one optic cross-section of the object, and
a control module for controlling the optic tomograph.

9. The optic tomograph according to claim 8, in which the optic tomograph is embodied as a microscope.

10. The optic tomograph according to claim 8, with the optic tomograph being embodied as a laser scanning microscope.
